(19) United States
(12) Patent Application Publication
(10) Pub. No.: US 2024/0298981 A1
(43) Pub. Date: Sep. 12, 2024

(54) MINI C-ARM WITH A VARIABLE APERTURE ASSEMBLY

(71) Applicant: Hologic, Inc., Marlborough, MA (US)

(72) Inventors: Tri PHAM, Marlborough, MA (US); Marc HANSROUL, Marlborough, MA (US); David PHILIPS, Marlborough, MA (US)

(73) Assignee: Hologic, Inc., Marlborough, MA (US)

(21) Appl. No.: 18/258,468

(22) PCT Filed: Jun. 9, 2021
(86) PCT No.: PCT/US2021/036619

§ 371 Date: Jun. 20, 2023

Related U.S. Application Data
(60) Provisional application No. 63/129,266, filed on Dec. 22, 2020.

(51) Int. Cl. A61B 6/40; A61B 6/00

(52) U.S. Cl. CPC A61B 6/4035; A61B 6/4405; A61B 6/4441; A61B 6/4476; A61B 6/5235

(57) ABSTRACT

A mobile imaging system or mini C-arm with a variable aperture assembly is disclosed. The mini C-arm includes a detector and a moveable source. The aperture assembly being operatively coupled to the moveable source. The aperture assembly including a plurality of independently controllable blades such as, for example, first, second, third, and fourth blades, to define a variable aperture through which an X-ray beam is passed from the source to the detector. In one embodiment, each of the plurality of blades is independently controlled. In one embodiment, the aperture assembly includes a PCB sensor aligned with the plurality of blades for detecting a position of each of the plurality of the blades.

### CROSS-REFERENCE TO RELATED APPLICATIONS

This is a non-provisional of, and claims the benefit of the filing date of, pending U.S. provisional patent application No. 63/129,266, filed Dec. 22, 2020, entitled “Mini C-arm with a Variable Aperture Assembly,” the entirety of which application is incorporated by reference herein.

### FIELD OF THE DISCLOSURE

The present invention generally relates to imaging systems, and, more particularly, to a mobile imaging system such as, for example, a mini C-arm, having a variable aperture assembly associated with an X-ray source for adjusting the X-ray field.

### BACKGROUND OF THE DISCLOSURE

Mini C-arms are mobile imaging systems that provide non-invasive means for imaging a patient's bone and/or tissue (collectively a patient's anatomy). A mini C-arm is a mobile fluoroscope that generally include a mobile base, an arm assembly, and a C-arm assembly including an X-ray source and a detector.

For example, referring to FIG. 1, a conventional embodiment of a mini C-arm 100 is shown. As illustrated, the mini C-arm 100 includes a base 120, a C-arm assembly 150, and an arm assembly 130 for coupling the C-arm assembly 150 to the base 120. As illustrated, the base 120 may include a platform 122 and a plurality of wheels 124 extending from a bottom surface of the platform 122 so that the base 120, and hence the mini C-arm 100, can be movably located by the operator as desired. The wheels 124 are selectably lockable by the operator so that when in a locked state, the wheels 124 allow the operator to manipulate the arm assembly 130 without shifting the location or orientation of the base 120. The base 120 may also include a cabinet 126. As will be appreciated by one of ordinary skill in the art, the cabinet 126 may be sized and configured for storing, for example, controls (not shown) for operating the mini C-arm 100, electrical components (not shown) needed for operation of the mini C-arm 100, counterweights (not shown) needed to balance extension of the C-arm assembly 150, a brake system, a cord wrap, etc. The cabinet 126 may also include, for example, a keyboard, one or more monitors, a printer, etc.

The arm assembly 130 may include a first arm 132 and a second arm 134, although it is envisioned that the arm assembly 130 may include a lesser or greater number of arms such as, for example, one, three, four, etc. The arm assembly 130 enables variable placement of the C-arm assembly 150 relative to the base 120. As illustrated in the exemplary embodiment, the arm assembly 130, and more specifically the first arm 132, may be coupled to the base 120 via a vertically adjustable connection. Other mechanisms for coupling the arm assembly 130 to the base 120 are also envisioned including, for example, a pivotable connection mechanism. The second arm 134 may be coupled to the first arm 132 via a joint assembly to enable the second arm 134 to move relative to the first arm 132. In addition, the second arm 134 may be coupled to the C-arm assembly 150 via an orbital mount 170, as will be described in greater detail below. Thus arranged, the arm assembly 130 enables the C-arm assembly 150 to be movably positioned relative to the base 120.

As previously mentioned, the mini C-arm 100 also includes a C-arm assembly 150. The C-arm assembly 150 includes a source 152, a detector 154, and an intermediate body portion 156 for coupling to the source 152 and the detector 154. As will be readily known by one of ordinary skill in the art, the imaging components (e.g., X-ray source 152 and detector 154) receive photons and convert the photons/X-rays to a manipulable electrical signal that is transmitted to an image processing unit (not shown). The image processing unit may be any suitable hardware and/or software system, now known or hereafter developed to receive the electrical signal and to convert the electrical signal into an image. Next, the image may be displayed on a monitor or TV screen. The image can also be stored, printed, etc. The image may be a single image or a plurality of images.

The intermediate body portion 156 of the C-arm assembly 150 includes a curved or arcuate configuration. For example, the intermediate body portion 156 may have a substantially “C” or “U” shape, although other shapes are envisioned. The intermediate body portion 156 includes a body portion 158 and first and second end portions 160, 162 for coupling to the source and detector 152, 154, respectively. In certain embodiments, the body portion 158 and the first and second ends 160, 162 of the intermediate body portion 156 may be integrally formed. Alternatively, these portions of the intermediate body portion 156 can be separately formed and coupled together. The X-ray source 152 and the detector 154 are typically mounted on opposing ends of the C-arm assembly 150 and are in fixed relationship relative to each other. The X-ray source 152 and the detector 154 are spaced apart by the C-arm assembly 150 sufficiently to define a gap between them in which the patient's anatomy can be inserted in the path of the X-ray beam. As illustrated, the C-arm assembly 150 may include an orbital mount 170 for coupling the C-arm assembly 150 to the arm assembly 130. The body portion 158 rotates or orbits relative to the orbital mount 170 to provide versatility in positioning the imaging components relative to the portion of the patient's anatomy to be irradiated.

In certain instances, it is desirable to obtain multiple X-ray views of a patient's anatomy. For example, during an orthopedic surgery on a patient's hand, wrist, elbow, foot, etc. a wide range of internal and/or external hardware devices such as, for example, bone plates, screw, pins, wires, etc. may be used (collectively referred to herein as orthopedic devices without the intent to limit). For these procedures, a surgeon may want to acquire multiple X-ray views at different angles to, for example, assess the depth of implant placement. With current technologies, this can be accomplished by removing the patient's anatomy from the detector and repositioning the imaging components relative to the patient and/or by changing the position of the patient's anatomy relative to the X-ray source and detector. These methods, which require moving the patient's anatomy, are undesirable particularly when performing surgeries or evaluating the extent of an injury.

It is with respect to these and other considerations that the present improvements may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is a mini C-arm imaging apparatus. In one embodiment, the mini C-arm imaging apparatus comprises a C-arm assembly, a movable base, and an arm assembly coupling the C-arm assembly to the movable base. The C-arm assembly includes a first end, a second end, and a curved intermediate body portion extending between the first and second ends. The C-arm assembly also includes an X-ray source adjacent the first end and a detector at the second end, the curved intermediate body portion defines an arc length extending between the first and second ends. The X-ray source is movable along the arc length of the curved intermediate body portion and relative to the detector to enable the mini C-arm to acquire a first image when the X-ray source is at a first position on the curved intermediate body portion and a second image when the X-ray source is at a second position on the curved intermediate body portion, the second position being different that the first position, so that the first and second images of the patient's anatomy are taken at different angles relative to the patient's anatomy and are acquired without moving the patient's anatomy. The C-arm assembly also includes an aperture assembly operatively coupled to the X-ray source such that an X-ray beam passes from the X-ray source through the aperture assembly and onto the detector. The aperture assembly comprises first, second, third, and fourth blades defining an aperture, the first and second blades positioned on opposing sides of the aperture, the third and fourth blades positioned on opposing sides of the aperture and orthogonal to the first and second blades, each of the first, second, third, and fourth blades being independently controlled to adjust a field of view.

In one embodiment, the aperture assembly includes first, second, third, and fourth motors operatively coupled to the first, second, third, and fourth blades, respectively.

In one embodiment, the aperture assembly further includes first, second, third, and fourth lead screws, the first lead screw coupling the first motor to the first blade, the second lead screw coupling the second motor to the second blade, the third lead screw coupling the third motor to the third blade, and the fourth lead screw coupling the fourth motor to the fourth blade.

In one embodiment, the aperture assembly includes a rotational motor and a drive belt, the drive belt extending between the rotational motor and a radial gear of the aperture assembly so that activation of the rotational motor rotates the aperture assembly relative to the X-ray source.

In one embodiment, the aperture assembly includes a position sensing system to detect movement of the X-ray source, upon detection of movement of the X-ray source, the rotational motor automatically rotates the aperture assembly to match movement of the X-ray source.

In one embodiment, the aperture assembly includes a plurality of bearings to ride in a corresponding groove formed in the aperture assembly to guide rotation of the aperture assembly.

In one embodiment, the aperture assembly includes a sensor to detect a position of each of the first, second, third, and fourth blades.

In one embodiment, the aperture assembly includes a first subassembly and a second subassembly, the first subassembly including the first and second blades, the second subassembly including the third and fourth blades.

In one embodiment, the sensor is an inductance sensor PCB positioned between the first subassembly and the second subassembly.

In one embodiment, the sensor PCB includes an upper surface, a lower surface, and first, second, third, and fourth coils, the first and second coils positioned in the upper surface and in alignment with the first and second blades, respectively, the third and fourth coils positioned in the lower surface and in alignment with the third and fourth blades, respectively, and wherein each of the first, second, third, and fourth blades includes a target such that movement of the blades causes a respective target to move relative to a respective coil creating a resulting electromagnetic field.

In one embodiment, the aperture assembly includes a pre-collimator and a filter to attenuate the incoming X-ray beam, the pre-collimator reducing a size of the incoming X-ray beam before passing through the aperture.

In one embodiment, the aperture assembly enables a custom magnification view to enable an operator to adjust the size of the X-ray beam emitted from the X-ray source to select a desired field of view.

In one embodiment, the first and second images of the patient's anatomy are different radiographic views of the patient's anatomy.

In one embodiment, the first and second images of the patient's anatomy are combined into a three-dimensional rendering of the patient's anatomy.

In an alternate embodiment, a method for generating a custom magnification image of a patient's anatomy using a mini C-arm is disclosed. In one embodiment, the mini C-arm includes an aperture assembly having independently controlled aperture blades. The method comprises taking an initial X-ray of the patient's anatomy positioned on a detector of the mini C-arm; selecting a region of interest of the patient's anatomy; adjusting the independently controlled aperture blades of the mini C-arm to focus an emitted X-ray beam to the selected region of interest; and digitally magnifying the selected region of interest to generate a magnified image of the patient's anatomy without removing the patient's anatomy from the detector.

In one embodiment, digitally magnifying the selected region of interest is performed automatically.

In one embodiment, digitally magnifying the selected region of interest is performed upon input from an operator.

In one embodiment, digitally magnifying the selected region of interest comprises magnifying the image by a percentage input by the user.

In one embodiment, digitally magnifying the selected region of interest comprises a percent magnification, the percent magnification being a pre-set magnification based on the anatomy being imaged.

In one embodiment, selecting a region of interest of the patient's anatomy comprises using a user-interface including a control panel operatively coupled to a computer system, the control panel including an array of switches or buttons to enable an operator to select the region of interest by cycling through sections of the initial X-ray or flipping through a series of images taken from the initial X-ray.

In one embodiment, selecting a region of interest of the patient's anatomy comprises a foot pedal operatively coupled to a computer system, the foot switches including an array of foot switches to enable an operator to cycle through different areas of the initial X-ray to select the region of interest.

In one embodiment, selecting a region of interest of the patient's anatomy comprises one of a keyboard, a touch screen, or a combination thereof, to enable an operator to select the region of interest.

In one embodiment, taking an initial X-ray of the patient's anatomy positioned on a detector of the mini C-arm comprises taking an image of a full-view of the patient's anatomy.

### BRIEF DESCRIPTION OF THE DRAWINGS

By way of example, a specific embodiment of the disclosed device will now be described, with reference to the accompanying drawings, in which:

FIG. 1 is a perspective view of a conventional mobile imaging system or mini C-arm;

FIG. 2 is a perspective view of an example embodiment of a C-arm assembly in accordance with one or more features of the present disclosure, the C-arm assembly may be used in connection with the mini C-arm shown in FIG. 1;

FIG. 3 is a perspective view of an example embodiment of a source module including an aperture assembly in accordance with one or more features of the present disclosure, the source module including the aperture assembly may be used in connection with the C-arm assembly shown in FIG. 2 and/or with the mini C-arm shown in FIG. 1;

FIG. 4A is a perspective view of an alternate example embodiment of a source module including an aperture assembly in accordance with one or more features of the present disclosure, the source module including the aperture assembly may be used in connection with the C-arm assembly shown in FIG. 2 and/or with the mini C-arm shown in FIG. 1;

FIG. 4B is an exploded perspective view of the source module and the aperture assembly shown in FIG. 4A;

FIG. 5A is a top perspective view of the aperture assembly shown in FIGS. 4A and 4B;

FIG. 5B is a bottom perspective view of the aperture assembly shown in FIG. 5A;

FIG. 5C is an exploded view of the aperture assembly shown in FIG. 5A;

FIG. 6A is a top perspective view of a first or upper subassembly of the aperture assembly shown in FIGS. 4A and 4B;

FIG. 6B is a bottom perspective view of the first or upper subassembly of the aperture assembly shown in FIG. 6A;

FIG. 6C is an exploded view of the first or upper subassembly of the aperture assembly shown in FIG. 6A;

FIG. 7A is a top perspective view of a second or lower subassembly of the aperture assembly shown in FIGS. 4A and 4B;

FIG. 7B is a bottom perspective view of the second or lower subassembly of the aperture assembly shown in FIG. 7A;

FIG. 7C is an exploded view of the second or lower subassembly of the aperture assembly shown in FIG. 7A;

FIG. 8A is a top perspective view of a sensor PCB that may be positioned between the first or upper subassembly and the second or lower subassembly of the aperture assembly in accordance with one or more features of the present disclosure;

FIG. 8B is a bottom perspective view of the sensor PCB shown in FIG. 8A;

FIG. 8C is a perspective view illustrating the sensor PCB shown in FIG. 8A positioned between the first or upper subassembly and the second or lower subassembly of the aperture assembly in accordance with one or more features of present disclosure;

FIG. 9A is a perspective view of an alternate example embodiment of an aperture assembly in accordance with one or more features of the present disclosure, the aperture assembly may be used in connection with the C-arm assembly shown in FIG. 2 and/or with the mini C-arm shown in FIG. 1;

FIG. 9B is a bottom perspective view of the aperture assembly shown in FIG. 9A;

FIG. 10 is a perspective view of an alternate example embodiment of an aperture assembly in accordance with one or more features of the present disclosure, the aperture assembly may be used in connection with the C-arm assembly shown in FIG. 2 and/or with the mini C-arm shown in FIG. 1;

FIG. 11 is a perspective view of an alternate example embodiment of an aperture assembly in accordance with one or more features of the present disclosure, the aperture assembly may be used in connection with the C-arm assembly shown in FIG. 2 and/or with the mini C-arm shown in FIG. 1;

FIG. 12A is a perspective view of an alternate example embodiment of an aperture assembly in accordance with one or more features of the present disclosure, the aperture assembly may be used in connection with the C-arm assembly shown in FIG. 2 and/or with the mini C-arm shown in FIG. 1;

FIG. 12B is a bottom perspective view of the aperture assembly shown in FIG. 12A; and

FIGS. 13A and 13B illustrate an example embodiment of a MAG view in accordance with one or more features of the present disclosure.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements.

### DETAILED DESCRIPTION

Numerous embodiments of an aperture assembly (e.g., a collimator) arranged and configured to be operatively coupled to a source of a mobile imaging system or mini C-arm (mobile imaging system or mini C-arm used interchangeably herein without the intent to limit) in accordance with the present disclosure will now be described more fully hereinafter with reference to the accompanying drawings, in which preferred embodiments of the present disclosure are presented. The aperture assembly of the present disclosure may, however, be embodied in many different forms and should not be construed as being limited to the embodiments set forth herein. Rather, these embodiments are provided so that this disclosure will convey certain example features of the aperture assembly to those skilled in the art.

As previously mentioned, in conventional mini C-arms, the source and detector are mounted on opposing ends of a C-arm assembly and are fixed relative to each other. As a result, while operators can move the C-arm assembly and the imaging components relative to the patient's anatomy to acquire an image of the patient's anatomy at a different angle, this requires removing the patient's anatomy from the detector and repositioning the imaging components relative to the patient and/or by changing the position of the patient's anatomy relative to the X-ray source and detector.

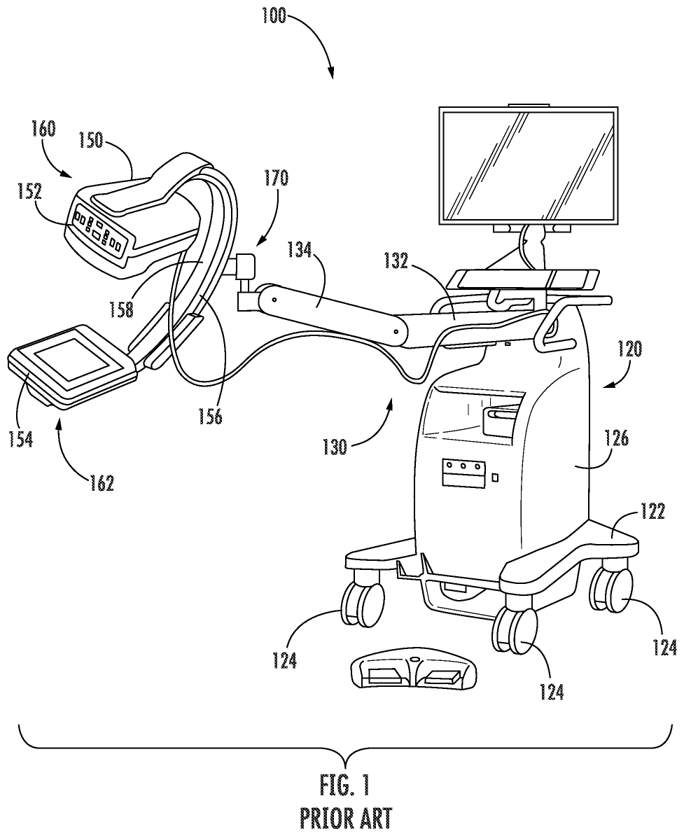

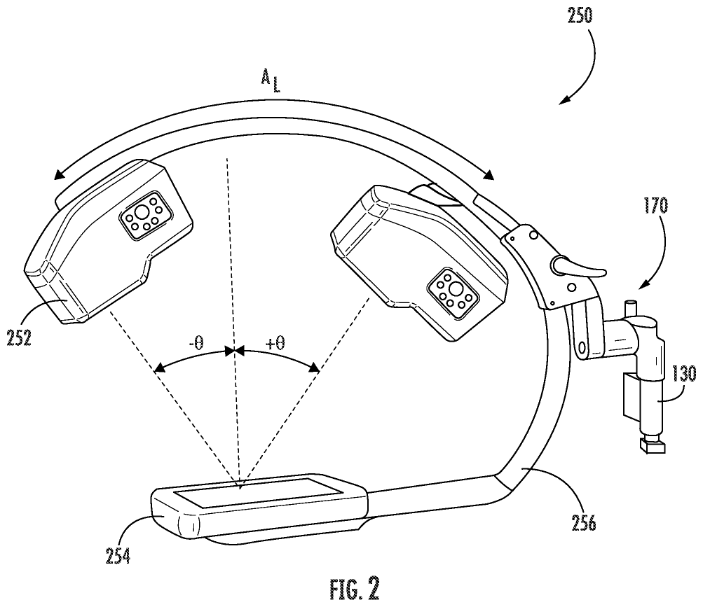

In accordance with one or more features of the present disclosure, the mini C-arms such as, for example, mini C-arm 100 shown in FIG. 1, includes a source movable relative to the intermediate body portion of the C-arm assembly and/or relative to the detector. For example, referring to FIG. 2, in one example embodiment in accordance with the present disclosure, the mini C-arm may include a C-arm assembly 250 including a source 252, a detector 254, and an intermediate body portion 256 wherein the source 252 is movable along an arc length A_{L} of the intermediate body portion 256 of the C-arm assembly 250. In one embodiment, the C-arm assembly 250 may enable ±θ degrees of movement of the source 252 relative to the detector 254. In one example embodiment, θ may be equal to 45 degrees.

In addition, and/or alternatively, the source may move in a plane transverse to the arc length A_{L}. In either event, the source 252 may be repositioned to, for example, enable the operator to acquire multiple images of the patient's anatomy without movement of the detector 254. More specifically, by arranging the source 252 to move along the arc length A_{L} of the intermediate body portion 256 of the C-arm assembly 250 (and/or transverse thereto), the surgeon can acquire multiple views of the patient's anatomy including, for example, an anterior-posterior view or a posteroanterior view (PA) and an oblique or lateral view without moving the patient's anatomy from the detector 254.

The source 252 may be movably coupled to the intermediate body portion 256 of the C-arm assembly 250 via any known mechanism for movably coupling the source 252 to the C-arm assembly 250. For example, in one example embodiment, the source 252 may be coupled to the intermediate body portion 256 of the C-arm assembly 250 via a track that extends along an arc length A_{L} thereof. The source 252 may be coupled to the track so that the source 252 can be moved, repositioned, etc., along the track, which extends along the arc length A_{L} of the intermediate body portion 256 of the C-arm assembly 250.

In one embodiment, the source 252 may be manually positionable along the arc length A_{L} of the intermediate body portion 256 of the C-arm assembly 250. For example, in one embodiment, the source 252 may slide along the arc length A_{L} of the intermediate body portion 256 of the C-arm assembly 250. In one embodiment, the source 252 can be continuously movable along an arc length A_{L} of the intermediate body portion 256 of the C-arm assembly 250. Alternatively, the source 252 may be positionable at predefined angles, positions, etc.

Alternatively, and/or in addition, in one embodiment, the source 252 may be moved relative to the intermediate body portion 256 of the C-arm assembly 250 via, for example, motorized control. For example, the mini C-arm may include a motor to move the source 252 along an arc length A_{L} of the intermediate body portion 256 of the C-arm assembly 250. For example, in one embodiment, the source 252 may be coupled to the intermediate body portion 256 of the C-arm assembly 250 via a connector unit, which may house a motor operatively coupled to an output gear, which may be operatively coupled to a drive system such as, for example, a drive belt and pulley system, a lead screw drive system, or the like. Activation of the motor rotates the output gear, which rotates the belt about the pulleys, which moves the source.

By incorporating motorized controls, movement of the source 252 can be better controlled thus facilitating precise acquisition of images at various angular positions. Thus arranged, the surgeon can generate the X-ray images from a large range of angles covering anterior-posterior views and oblique/lateral views. In addition, as will be described in greater detail below, when utilizing a mini C-arm with MAV and/or TOMO imaging qualities, utilization of motorized controls becomes more important since precise control of the speed and angle of the images is needed.

As previously mentioned, the source may be movable, repositionable, etc. relative to the detector by any suitable mechanism now known or hereafter developed. In one embodiment, the mini C-arm enables the operator to move, reposition, etc., the X-ray source or X-ray source module (terms used interchangeably without the intent to limit or distinguish) along an arc length of the C-arm assembly. In addition, and/or alternatively, in one embodiment, the detector may be rotatable about an axis passing perpendicular to a face of the detector. In addition, and/or alternatively, in one embodiment, the source may be movable along an arc perpendicular to the arc length of the C-arm assembly. Additional details regarding mechanisms for moving the source and rotating the detector can be found in U.S. patent application No. (Attorney Docket Number 8105.0023Z and 8105.0023Z2) entitled “Mini C-arm with Movable Source and/or Detector”, the entire contents of each application incorporated herein by reference it their entirety.

In accordance with one or more features of the present disclosure, by enabling the source to be movable relative to the detector, the mini C-arm enables surgeons to acquire multiple X-ray images, views, etc. The X-ray images may be acquired at different or various positions and/or angles. For example, multiple X-ray images may be acquired to provide different views and/or multiple X-ray images may be acquired and then manipulated/combined into a three-dimensional model or rendering. Moreover, for example, X-ray images can be acquired at different viewing angles during a drilling procedure to provide continuous orthopedic device positioning information and hence allow surgeons to correct their position, insertion angle, depth, etc. in real-time. Thus arranged, the mini C-arm facilitates improved assessment of the orthopedic devices during the surgical procedure with reduce retake rates, reduce risks of post-operative complications, and improved overall intervention time and quality.

In one embodiment, a first X-ray image may be taken in an anterior-posterior or posteroanterior angle while a second X-ray image may be taken in a lateral or oblique angle. In addition, in one embodiment, separate X-ray images may be combined to create a three dimensional volume or rendering of the patient's anatomy. Alternatively, acquisition of continuous X-ray images between a first image and a last image may be provided.

In accordance with one or more features of the present disclosure, by enabling the X-ray source or X-ray source module to move relative to the detector, the mini C-arm enables multi-angle view (MAV) and/or tomosynthesis (TOMO) image acquisition. MAV and TOMO imaging acquisition methods involve acquiring fluoroscopic images of the patient's static anatomy while the angle of the X-ray beam from the source to the image plane of the detector is varied (e.g., the angle between the X-ray source beam and the detector image plane may be varied while the center of the X-ray source beam remains aligned with the center of the detector's image plane throughout the range of relative movement between the x-ray source and the detector). With TOMO, the X-ray source moves in an arc over the detector through a limited angle range to capture multiple images of the patient's anatomy from different angles. TOMO image acquisition may involve continuous acquisition over the angle range, which can be, for example, forty degrees (e.g., ±20 degrees from a center of the arc length of the intermediate body portion of the C-arm assembly or relative to imaging axis, e.g., axis passing thru the X-ray source and detector when the X-ray source is aligned directly over the detector, as will be described in greater detail herein), with exposures made every 1 degree or so during the scan. These images are then reconstructed or “synthesized” into a set of three-dimensional images by a computer. With MAV image acquisition, the X-ray source is movable to acquire two or more images including off-axis views of the patient's anatomy (e.g., an oblique view or a lateral view).

In certain embodiments, MAV image acquisition and TOMO image acquisition may utilize substantially the same process. That is, the mini C-arm enables a plurality of images at various views, projections, angles, etc. to be acquired. However, the image processing and display may differ between the two modes (e.g., MAV image acquisition mode and TOMO image acquisition mode). For example, in connection with MAV, the images may be displayed side-by-side illustrating two separate 2D images acquired at different angles. Meanwhile, with TOMO, a 3D reconstructed image may be generated and then displayed. Both MAV and TOMO may also display the full sequence of images acquired (e.g., 2D Cine-type image).

In either event, in order to acquire multiple angles or views of the patient's anatomy without moving the patient's anatomy (e.g., it is preferred to maintain the patient's anatomy static in relationship to the detector as images are acquired to reduce motion-blur imaging effects), it is preferable to move the X-ray source relative to the patient's anatomy and/or the detector during the image acquisition workflow. For mini C-arms, the distance from the X-ray source to the detector's image plane (SID) cannot exceed 45 cm. As such, the SID needs to be controlled as the X-ray source moves through its MAV/TOMO angle ranges (e.g., distance can vary slightly with limited compromise to image quality). That is, during movement of the X-ray source, control over the source movement must be controlled to maintain the SID (e.g., precise control over the X-ray source movement is desirable to control the SID so it does not exceed 45 cm).

Additional details regarding MAV/TOMO imaging techniques can be found in International PCT Application No. (Attorney Docket Number 8105.0023WO) entitled “Mini C-arm with Movable Source”, the entire contents of each application incorporated herein by reference it their entirety.

Moreover, in order to permit acquisition of multiple images and/or to ensure high quality images when, or while, the source is being moved relative to the detector, in accordance with one or more features of the present disclosure, the X-ray source may include an aperture assembly to adjust the field of the X-ray beam emitted from the X-ray source, which is referred to as the field of view (FOV). In one embodiment, and as will be described in greater detail herein, the aperture assembly includes a plurality of aperture blades (e.g., first, second, third, and fourth aperture blades) for controlling, adjusting, etc., the size and/or shape of the beam area (field of view) projected onto the detector surface. In accordance with one or more features of the present disclosure, the aperture assembly enables each of the plurality of aperture blades to be independently controlled and thus variably positioned to enable complete freedom over the beam area (field of view) projected onto the detector surface.

For example, the aperture assembly controls the collimator's aperture size while the X-ray source module travels through its full range of motion. In one embodiment, while the X-ray source module angle changes, the C-arm control board adjusts the position of each aperture blade based upon the angle position of the source module. For example, in one embodiment, the operator may initiate image acquisition in either MAV or TOMO mode. The subsystem may sense, read, etc. the angle position of X-ray source module. That is, sensors may be utilized to sense the angle position of the source module and the information may be transferred to firmware and/or software. Next, the collimator blade positions (e.g., the blades in the aperture assembly) and angle of the source module may be compared using, for example, a transfer function or look-up table with interpolation. Sensors may be used to read the blade positions. For example, inductive sensors may be used. A command may then be sent to the collimator blade motors to adjust their position. The blade positions may then be adjusted and sensed/read to confirm their positioning. Steps may be repeated until the source module reaches its final position and/or angle for MAV/TOMO. Finally, the blade motor command may be stopped.

In addition, and/or alternatively, in accordance with one or more features of the present disclosure and as will be described in greater detail below, the aperture assembly may include a custom zoom or magnification scan option. For example, by utilizing and/or incorporating the aperture assembly, the mini C-arm enables the operator to adjust the area or field of the X-ray beam emitted from the X-ray source to limit or focus the projected beam onto the surface of the detector to focus on a certain area of interest. Thereafter, the area of interest can be magnified using a magnification option. The percentage magnified can either be customized or pre-set based on the region of interest.

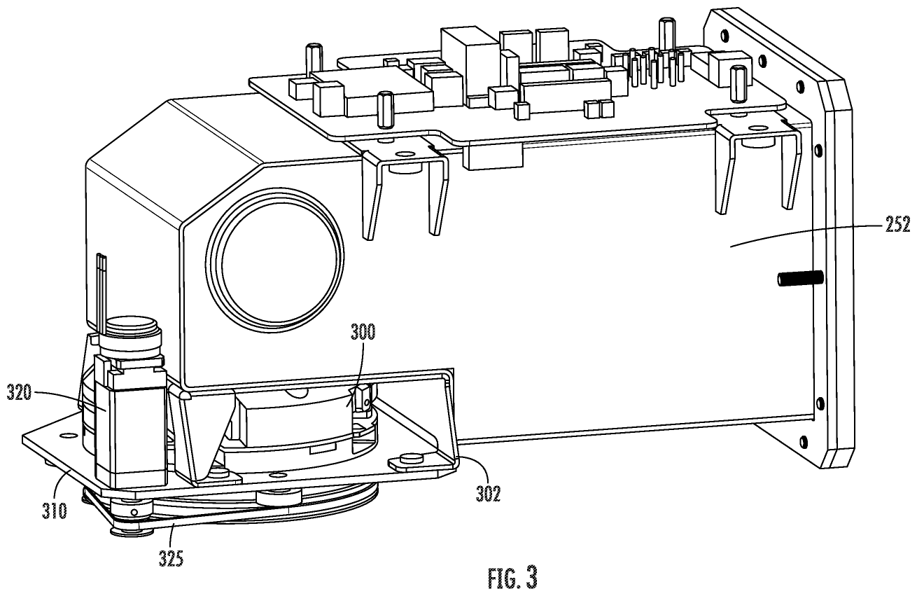

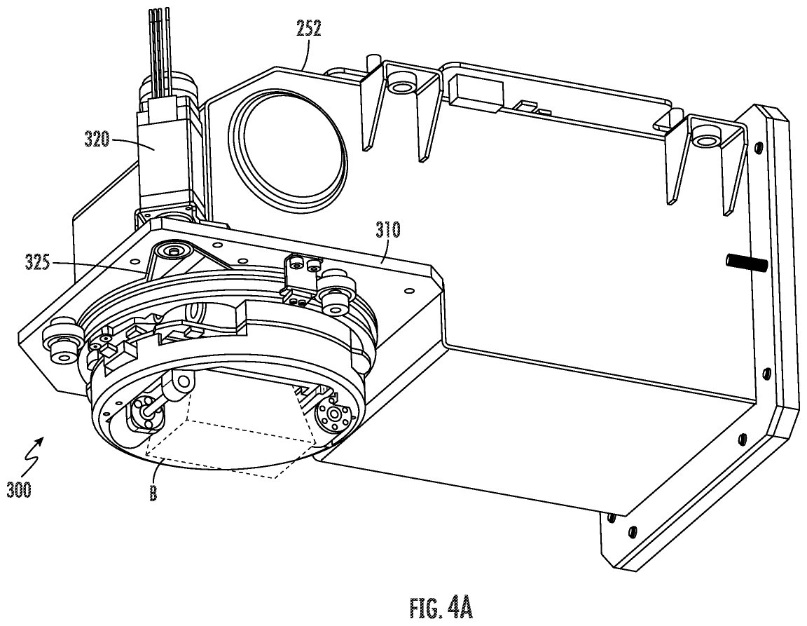

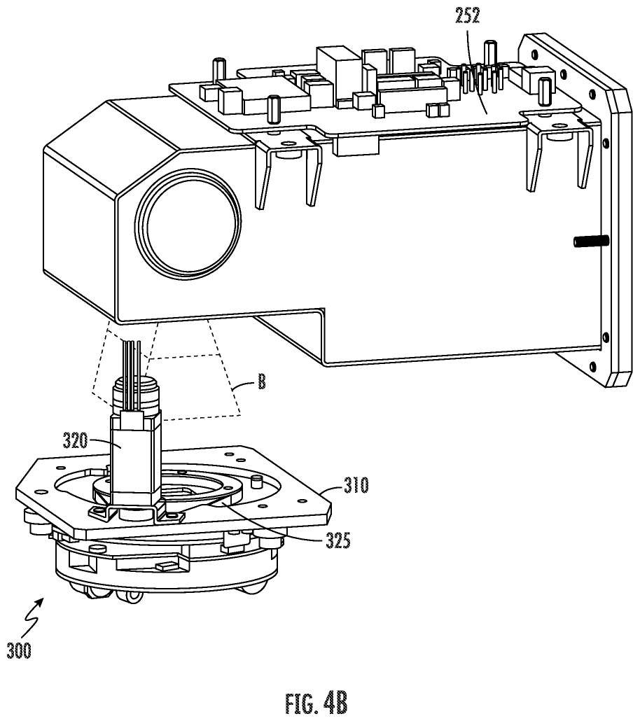

Referring to FIGS. 3-4B, example embodiments of an aperture assembly 300 are disclosed. As illustrated, the aperture assembly 300 is coupled to the X-ray source or source module such as, for example, the X-ray source 252 shown in FIG. 2. As illustrated, the aperture assembly 300 receives the X-ray beam B from the source 252 and enables the X-ray beam B to pass therethrough. As will be discussed in greater detail below, the aperture assembly 300 may alter, modify, etc. the shape of the X-ray beam B transmitted from the source 252 and passing through the aperture assembly 300, which in turn alters the beam area (field of view) projected onto the detector surface.

The aperture assembly 300 may be coupled to the source 252 via any suitable mechanism and/or method now known or hereafter developed. For example, in one embodiment, as illustrated in FIG. 3, the source 252 may include a bracket 302 for coupling to a mounting plate 310 of the aperture assembly 300 via one or more fasteners. As illustrated, in the embodiment of FIG. 3, the coupling mechanism (e.g., bracket and fastener) may be a bottom mount design (e.g., the bracket 302 is coupled to a mounting plate 310 positioned at or adjacent to a bottom portion of the aperture assembly 300). Alternatively, for example, referring to FIGS. 4A and 4B, the aperture assembly 300 may include a mounting plate 310 for coupling to the source 252 via one or more fasteners. As illustrated, in the embodiment of FIGS. 4A and 4B, the mounting plate 310 may be positioned at or adjacent to an upper end of the aperture assembly 300 thus the aperture assembly 300 may be referred to as a top mount design.

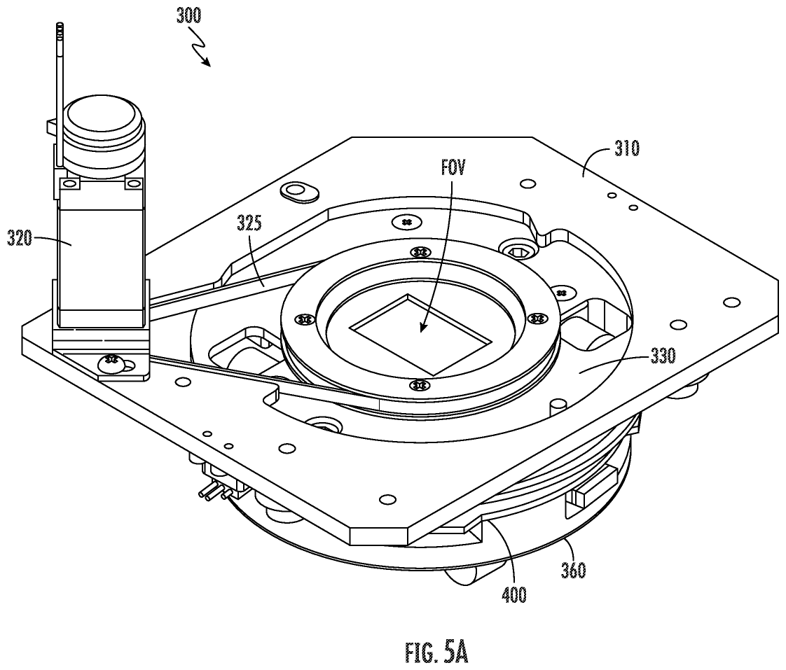

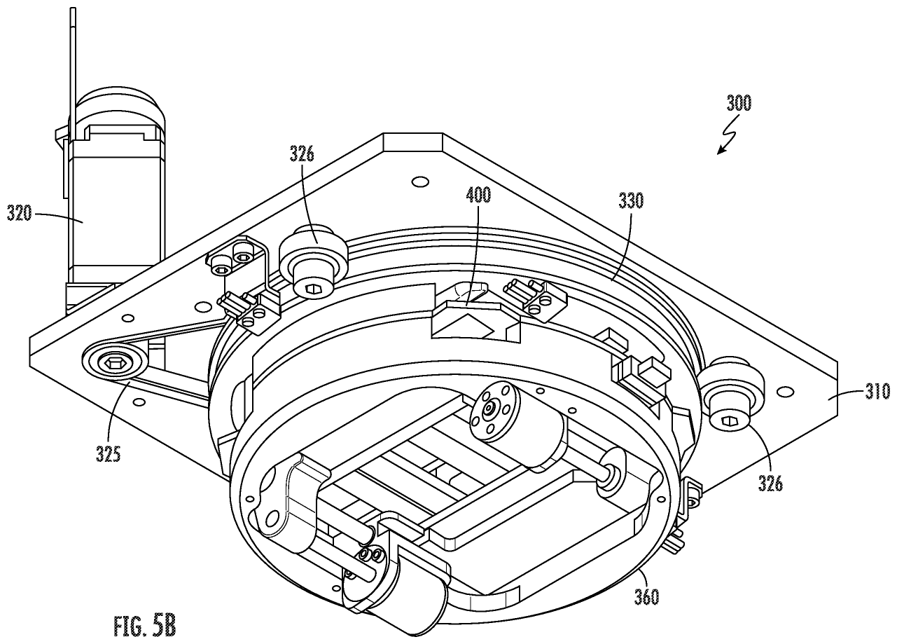

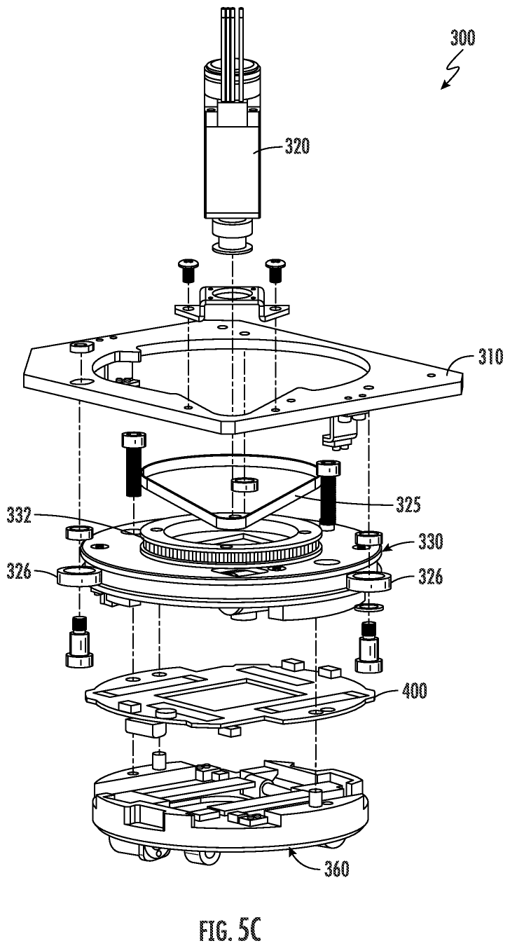

In either event, as will be described in greater detail below and with additional reference to FIGS. 5A-5C, the aperture assembly 300 may be rotatably coupled to the source 252. The aperture assembly 300 may be rotatably coupled to the source 252 via any suitable mechanism and/or method now known or hereafter developed. For example, in one embodiment, as illustrated in FIGS. 3-5C, the aperture assembly 300 may include a motor 320 operatively coupled to the aperture assembly 300 so that activation of the motor 320 rotates the aperture assembly 300. For example, as illustrated, the motor 320 may be coupled to the aperture assembly 300 via a drive belt and pulley system 325. However, alternate mechanisms for coupling the motor 320 to the aperture assembly 300 are envisioned. For example, as will be described in greater detail below in connection with an alternate embodiment, the motor may be coupled to the aperture assembly via a gear driven system.

The aperture assembly 300 may include and/or be operatively associated with a position sensing system so that the aperture assembly 300 can be automatically rotated to match movement, rotation, etc. of the detector 254. That is, for example, the operator may position (e.g., manually position) the detector 254 in a desired position relative to the patient's anatomy. In response, the aperture assembly 300 may automatically rotate as needed to match the position of the detector 254. For example, in one embodiment, after manual rotation of the detector 254 has been detected by the position sensing system, the motor 320 may be automatically activated so that the aperture assembly 300 is rotated to match the rotation of the detector 254. The position sensor system may be any suitable position sensing system now known or hereafter developed. For example, in one embodiment, the sensor may be a hall-effect sensor. Alternatively, however, other sensing systems may be utilized such as, for example, potentiometers, an inductance sensor, etc.

In addition, as illustrated, the aperture assembly 300 may include a plurality of bearings 326 to ride in a corresponding groove formed in the aperture assembly 300 to guide rotation of the aperture assembly 300. In one embodiment, one of the bearings 326 may be an eccentric bearing to assist with removing any gaps or slop during rotation of the aperture assembly 300. Incorporation of the bearings 326 assist in defining, maintaining, etc. the central rotational axis of the aperture assembly 300.

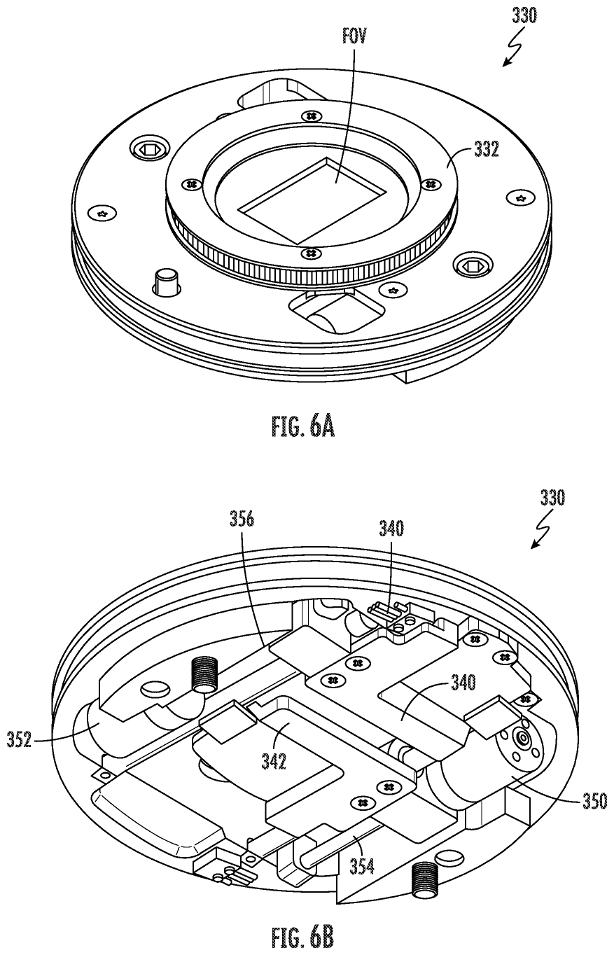

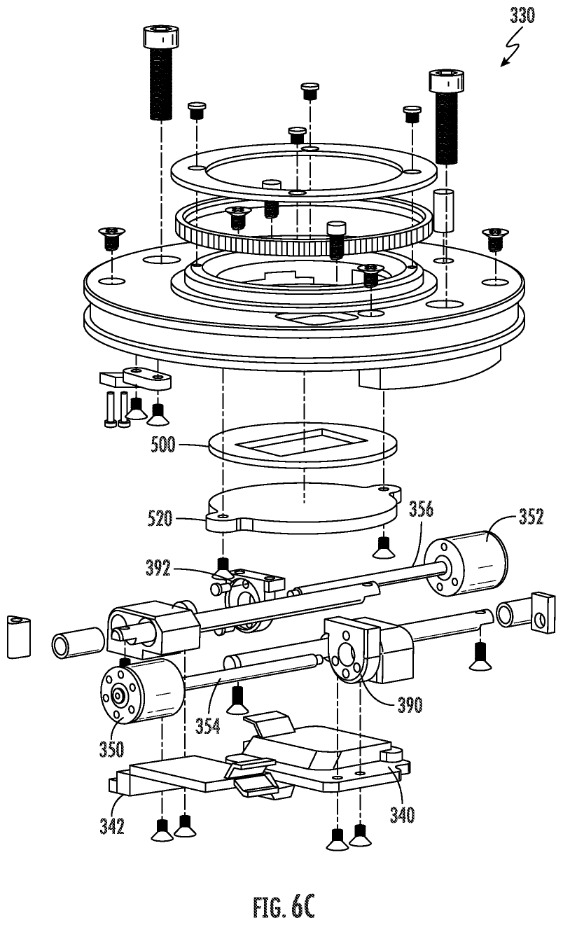

Referring to FIG. 6C, the aperture assembly 300 may include a pre-collimator 500 and an HVL filter 520. The filter 520 may be, for example, an aluminum filter, to attenuate the incoming X-ray beam B to ensure that the beam B is distributed evenly across the surface of the detector 254 (e.g., even intensity). The pre-collimator 500 may reduce the size of the incoming X-ray beam B before passing through the aperture (as will be described below) of the aperture assembly 300. In one embodiment, the size of the aperture of the pre-collimator 500 is slightly larger than the largest aperture of the aperture assembly 300 (e.g., the largest beam area (field of view) provided by the blades of the aperture assembly 300). Thus arranged, the X-ray beam B from the source 252 passes into the aperture assembly 300. The X-ray beam B passing initially through the pre-collimator 500 to initially reduce the size of the X-ray beam B before passing through the blades of the aperture assembly 300.

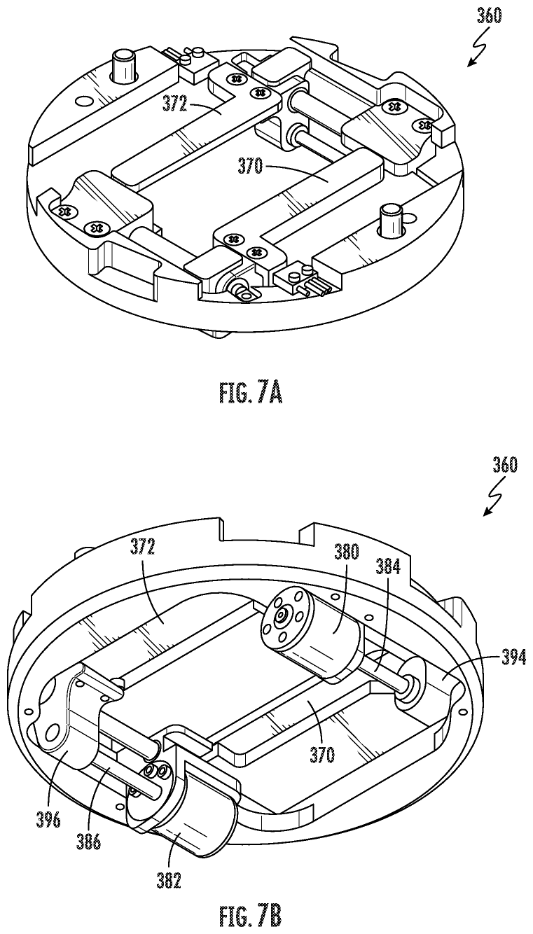

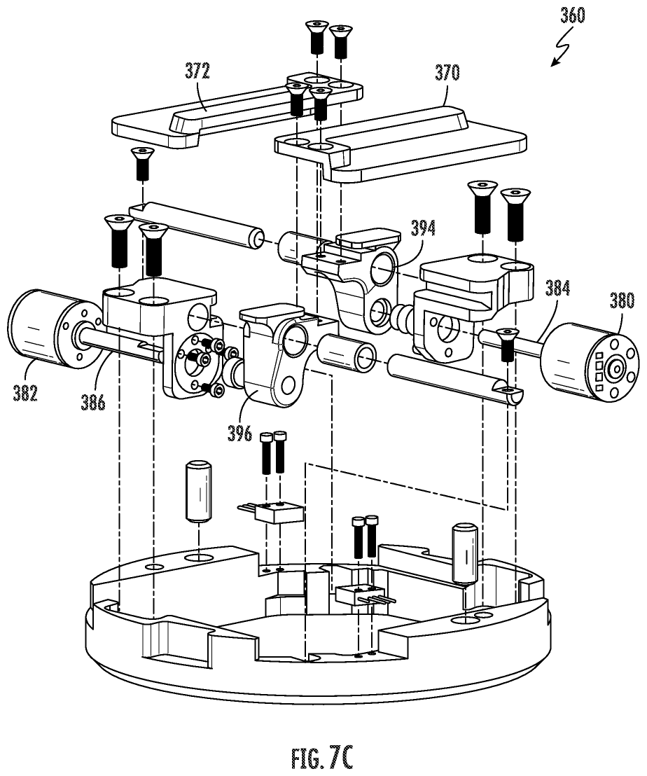

Referring to FIGS. 5A-5C, and with additional reference to FIGS. 6A-7C, the aperture assembly 300 may include a first or upper subassembly 330 and a second or lower subassembly 360. In addition, as will be described in greater detail below, in one embodiment, the aperture assembly 300 may include a sensor PCB 400 positioned between the first or upper subassembly 330 and the second or lower subassembly 360.

As illustrated, in the example embodiment, the first or upper subassembly 330 may be operatively coupled to the drive belt and pulley system 325. For example, as illustrated, in one embodiment, the first or upper subassembly 330 may include a radial gear 332 for receiving, interacting with, etc. the drive belt.

In one embodiment, as best illustrated in FIGS. 6B and 6C, the first or upper subassembly 330 includes first and second blades 340, 342. The first and second blades 340, 342 are positioned on opposing sides of the aperture. In addition, as best illustrated in FIGS. 7A-7C, the second or lower subassembly 360 also includes a pair of blades (e.g., third and fourth blades 370, 372) positioned on opposing sides of the aperture. The first and second blades 340, 342 of the first or upper subassembly 330 are positioned orthogonal relative to the third and fourth blades 370, 372 of the second or lower subassembly 360. Thus arranged, in one embodiment, each side of the aperture can be independently adjusted (e.g., each side of the rectangle or square defining the aperture can be independently adjusted as needed by repositioning one or more of the blades 340, 342, 370, 372). Thus arranged, the shape of the aperture defined by the blades 340, 342, 370, 372 can be shaped to substantially correspond with the shape of the detector. In one embodiment, when the aperture as defined by the blades 340, 342, 370, 372 is at its maximum size, the field of view provided by the aperture assembly 300 may be slightly larger than the detector size. Thereafter, by adjusting the positions of the blades 340, 342, 370, 372, the size and shape of the aperture can be adjusted as desired (e.g., as provided herein, each of the blades 340, 342, 370, 372 can be independently moved to change the size and shape of the aperture). For example, the blades 340, 342, 370, 372 can be adjusted between a rectangular field of view, a square field of view, etc. As will be appreciated, by independently adjusting the position of the blades 340, 342, 370, 372 the X-ray beam emitted from the X-ray source can be adjusted, limited, etc. as desired. By adjusting the position of the blades 340, 342, 370, 372, the operator can adjust where the X-ray beam falls on the surface of the detector and the size of the field of view. In certain embodiments, the blades 340, 342, 370, 372 can be simultaneously adjusted.

The blades 340, 342, 370, 372 may have any suitable shape and/or configuration. Movement of the blades 340, 342, 370, 372 alters the shape of the X-ray beam B transmitted from the source 252 and passing through the aperture assembly 300, which in turn alters the beam area (field of view) projected onto the detector surface. In one embodiment, the first and second blades 340, 342 of the first or upper subassembly 330 are shorter in length than the third and fourth blades 370, 372 of the second or lower subassembly 360. The stroke length of the first and second blades 340, 342 of the first or upper subassembly 330 may be smaller than the stroke length of the third and fourth blades 370, 372 of the second or lower subassembly 360. In one embodiment, each of the blades 340, 342, 370, 372 may have an L shape, although other shapes are envisioned. A portion (e.g., rectangular portion) of the blades 340, 342, 370, 372 may define the size of the aperture, the remaining portions of the blades 340, 342, 370, 372 prevent interference of any overlapping blades 340, 342, 370, 372 with the coils on the sensor PCB 400 (e.g., sensor PCB 400 including first, second, third, and fourth coils 406, 408, 410, 412, as will be described in greater detail below).

The blades 340, 342, 370, 372 may be manufactured from any suitable material that allows the blades to substantially prevent X-rays from passing through the blades. In addition, the blades can also be of any suitable thickness to prevent X-rays from passing therethrough. In one embodiment, and without limitation, the blades of the aperture assembly may be formed from a plastic such as a tungsten polymer material.

In one embodiment, as illustrated, each blade 340, 342, 370, 372 may be operatively coupled to a motor 350, 352, 380, 382 via a lead screw 354, 356, 384, 386 (e.g., each blade 340, 342, 370, 372 may include an internally threaded bore or nut for engaging an externally threaded lead screw 354, 356, 384, 386). Thus arranged, activation of one or more motors 350, 352, 380, 382 causes its respective lead screw 354, 356, 384, 386 to rotate, which adjusts the linear position of its respective blade 340, 342, 370, 372 along a longitudinal length of its respective lead screw 354, 356, 384, 386, thereby adjusting the shape of the X-ray beam B transmitted from the source 252 and passing through the aperture assembly 300, which in turn alters the field of view (e.g., moving the respective blades 340, 342, 370, 372 causes the spacing or opening between the respective blades 340, 342, 370, 372 to be adjusted thereby adjusting the shape of the X-ray beam B passing through the aperture assembly 300 and the beam area projected onto the detector surface). In one embodiment, the motors 350, 352, 380, 382 may be a stepper motor, although other suitable motors and corresponding drive systems can be utilized.

Thus arranged, in one embodiment, the aperture assembly 300 enables independent control of each blade 340, 342, 370, 372 to provide infinite adjustment over the beam area projected onto the detector surface. For example, by independently controlling each blade 340, 342, 370, 372, the left, right, top, and bottom sides of the size and/or shape of the X-ray beam B passing through the aperture assembly 300 can be independently adjusted. As such, as the X-ray source 252 is moved during, for example, MAV or TOMO imaging, as the source 252 is moved or rotated about the intermediate body portion 256 of the C-arm assembly 250, the X-ray beam B shifts relative to the detector 254. In accordance with the features of the present disclosure, the size and/or shape of the X-ray beam B passing through the aperture assembly 300 (e.g., collimator) can be adjusted to correspond with the repositioned source 252. For example, in connection with MAV imaging, the aperture assembly 300 enables multiple images of the patient's anatomy to be taken at two or more angular positions. For TOMO, an image reconstruction software may be used to create a three-dimensional rendering or volume of the patient's anatomy.

As previously mentioned, the aperture assembly 300 may include a sensor for detecting the position of each blade 340, 342, 370, 372. The sensor may be any suitable sensor now known or hereafter developed such as, for example, an encoder, a hall-effect sensor, an inductance sensor, etc. In one embodiment, the sensor may be an inductance sensor. For example, as illustrated in FIG. 5C, in one embodiment, the aperture assembly 300 may include an inductance sensor PCB 400 positioned between the first or upper subassembly 330 and the second or lower subassembly 360. The sensor PCB 400 monitors, determines, detects, etc. the position of each of the individual blades 340, 342, 370, 372.

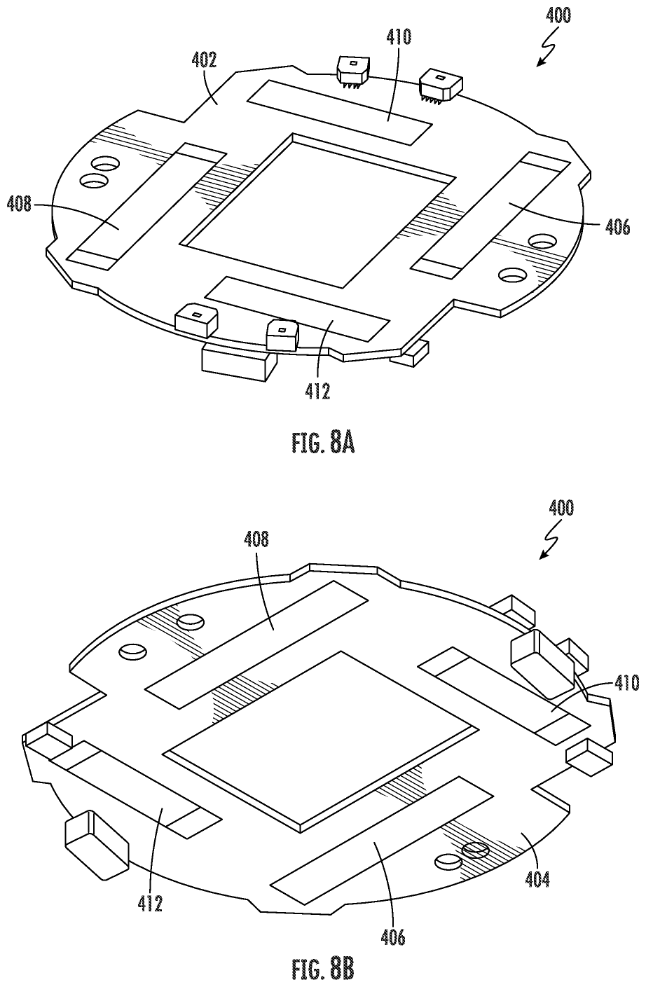

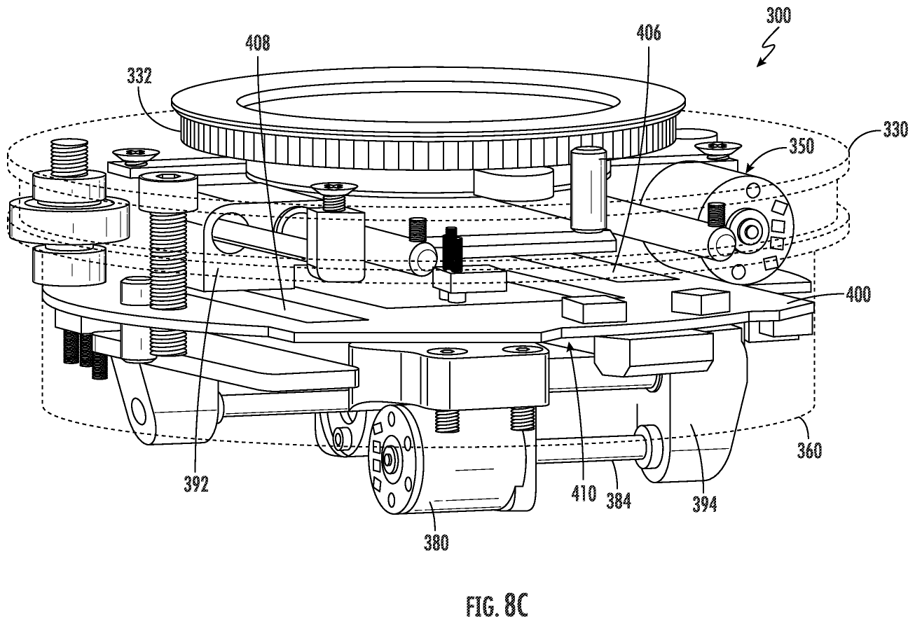

In one embodiment, referring to FIGS. 8A-8C, the sensor PCB 400 includes an upper surface 402 and a lower surface 404. In addition, the sensor PCB 400 includes first, second, third, and fourth coils 406, 408, 410, 412. As illustrated, the first and second coils 406, 408 are positioned in the upper surface 402. The third and fourth coils 410, 412 are positioned in the lower surface 404. Thus arranged, with the sensor PCB 400 positioned between the first or upper subassembly 330 and the second or lower subassembly 360, the first and second coils 406, 408 are positioned in alignment with the first and second blades 340, 342. Similarly, the third and fourth coils 410, 412 are positioned in alignment with the third and fourth blades 370, 372. Referring to FIG. 8C, each of the blades 340, 342, 370, 372 includes, or is operatively associated with, a target (e.g., a metal target) 390, 392, 394, 396. Thus arranged, activation of the motors 350, 352, 380, 382 rotates the lead screws 354, 356, 384, 386, which moves the blades 340, 342, 370, 372 to adjust the size and/or shape of the X-ray beam B passing through the aperture assembly 300. In addition, rotation of the lead screws 354, 356, 384, 386 moves the metal targets 390, 392, 394, 396, which creates a resulting electromagnetic field. By detecting the electromagnetic field via the coils 406, 408, 410, 412 in the sensor PCB 400, the position of each blade 340, 342, 370, 372 can be accurately determined (e.g., sensor PCB 400 detects the position of the target 390, 392, 394, 396 associated with each blade 340, 342, 370, 372 as the blades 340, 342, 370, 372 move across the coils 406, 408, 410, 412 positioned on the sensor PCB 400).

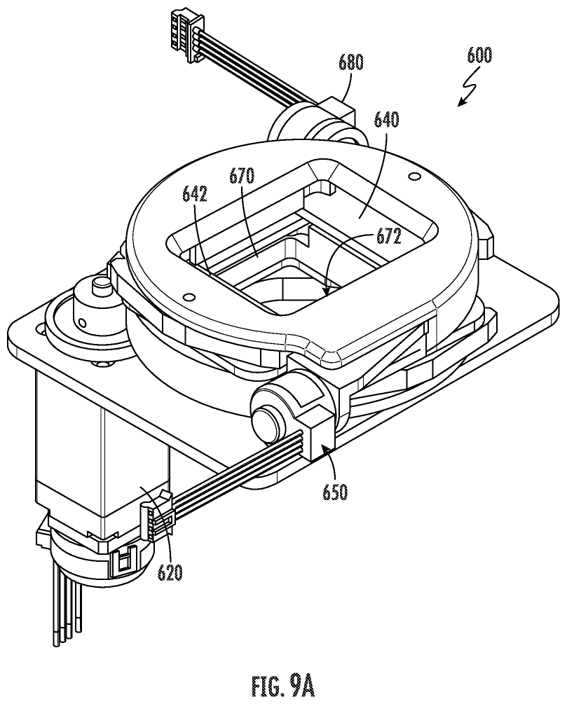

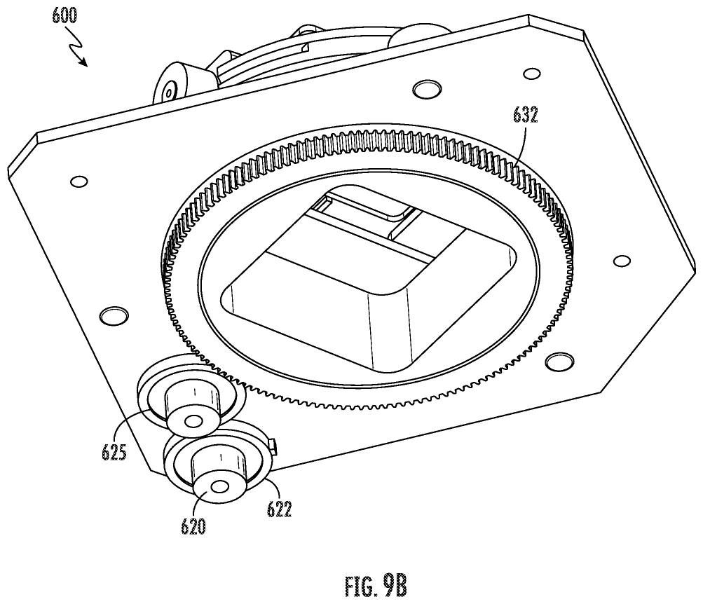

Referring to FIGS. 9A-12B, alternate embodiments of an aperture assembly are illustrated. As will be appreciated the aperture assemblies are substantially similar to the aperture assembly 300 described above. Thus for the sake of brevity, discussion of similar components are not repeated here. However, in contrast to aperture assembly 300 described above, alternate mechanisms for rotating the aperture assembly, and/or alternate mechanisms for moving the blades, and/or alternate mechanisms for determining, detecting, etc. the position of the blades are illustrated. As will be appreciated, the various mechanisms may be mixed and matched between the various embodiments of the aperture assemblies.

Referring to FIGS. 9A and 9B, an alternate embodiment of an aperture assembly 600 is illustrated. As illustrated, the aperture assembly 600 includes a motor 620 for enabling rotation of the aperture assembly 600. However in contrast to motor 320, motor 620 may be coupled to the aperture assembly 600 via a gear driven system (e.g., one or more interconnecting gears) 625. For example, an output gear 622 coupled to the motor 620 may be coupled to a spur gear 625, which may be coupled to a radial gear 632 associated with the aperture assembly 600.

Moreover in contrast to the aperture assembly 300, aperture assembly 600 may include first and second motors 650, 680 for controlling blades 640, 642, 670, 672. As such, left and right blades 640, 642 move in unison via activation of motor 650. Similarly, top and bottom blades 670, 672 move in unison via activation of motor 680. Thus arranged, in contrast to aperture assembly 300, aperture assembly 600 does not enable independent adjustment of all four blades.

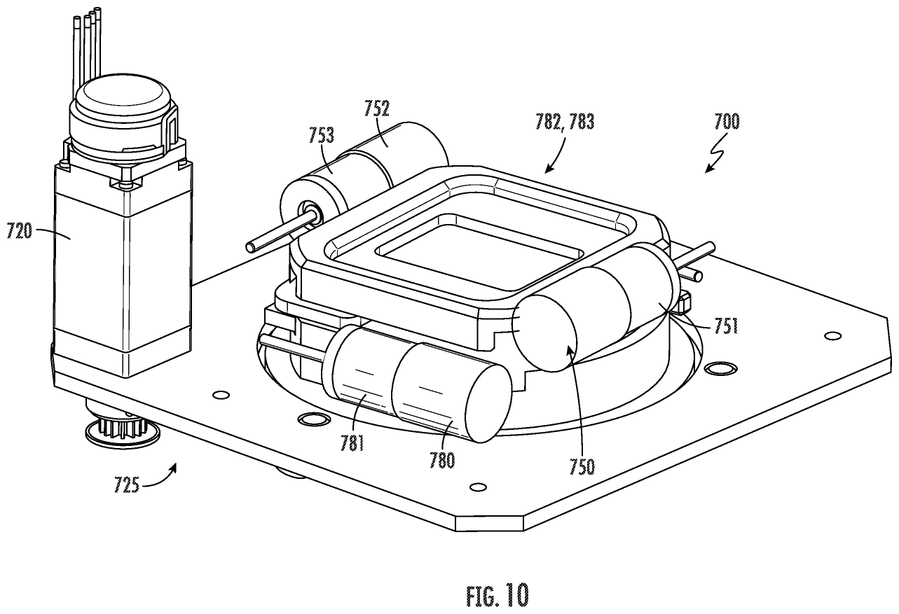

Referring to FIG. 10, an alternate embodiment of an aperture assembly 700 is illustrated. As illustrated, the aperture assembly 700 includes a motor 720 for enabling rotation of the aperture assembly 700. Similar to aperture assembly 300 described above, the motor 720 may be coupled to the aperture assembly 700 via a drive belt and pulley system 725, although alternate coupling mechanisms such as, for example, a gear driven system (e.g., one or more interconnecting gears) may be utilized. In addition, similar to aperture assembly 300 described above, the aperture assembly 700 may include first, second, third, and fourth motors 750, 752, 780, 782 for individually controlling each blade. In contrast to aperture assembly 300 described above, each motor 750, 752, 780, 782 may include an encoder 751, 753, 781, 783 associated therewith to measure, detect, etc. the position of each corresponding blade. As such, the sensor PCB 400 may be omitted.

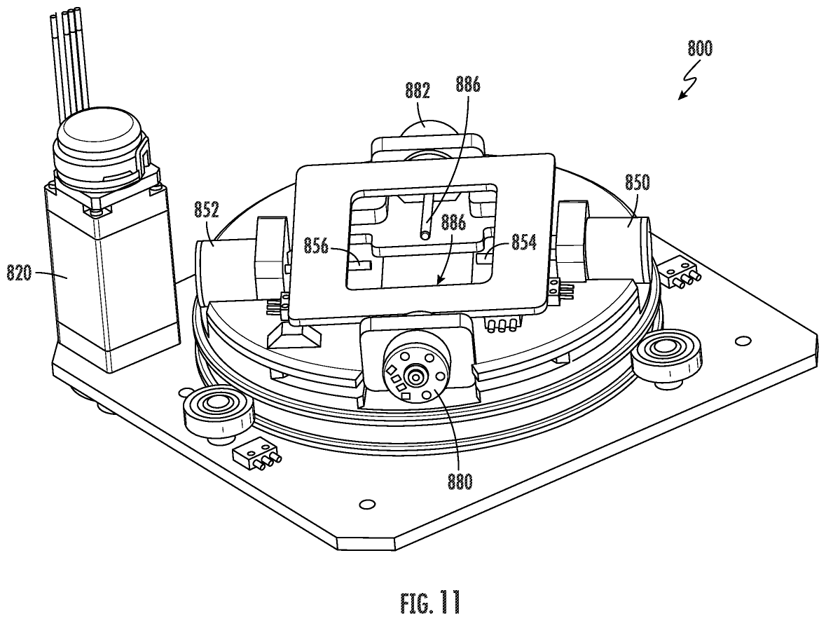

Referring to FIG. 11, an alternate embodiment of an aperture assembly 800 is illustrated. As illustrated, the aperture assembly 800 includes a motor 820 for enabling rotation of the aperture assembly 800. Similar to aperture assembly 600 described above, the motor 820 may be coupled to the aperture assembly 800 via a gear driven system (e.g., one or more interconnecting gears), although alternate coupling mechanisms such as, for example, a drive belt and pulley system may be utilized. In addition, similar to aperture assembly 300 described above, the aperture assembly 800 may include first, second, third, and fourth motors 850, 852, 880, 882 for individually controlling each blade. In contrast to aperture assembly 300 described above, each motor 850, 852, 880, 882 may include a lead screw 854, 856, 884, 886 that may be positioned parallel to length of travel of each blade. Thus arranged, similar to aperture assembly 300, aperture assembly 800 enables independent adjustment of all four blades. As illustrated, the sensor PCB may alternatively sense from a single side of the PCB.

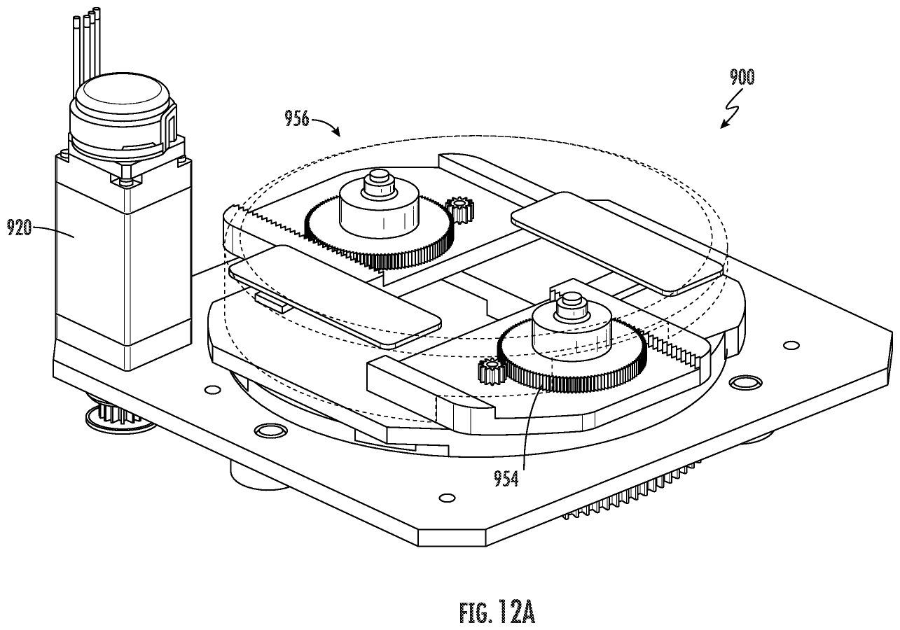

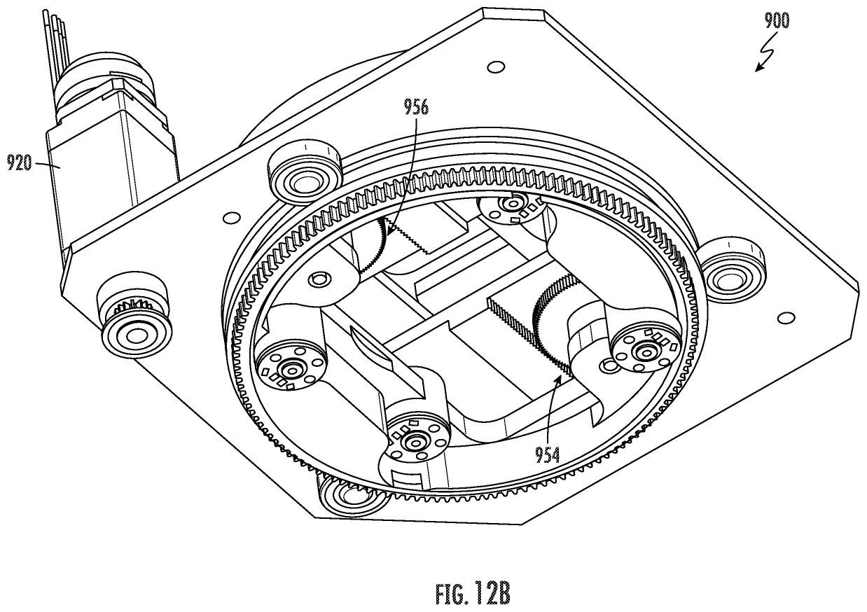

Referring to FIGS. 12A and 12B, an alternate embodiment of an aperture assembly 900 is illustrated. As illustrated, the aperture assembly 900 includes a motor 920 for enabling rotation of the aperture assembly 900. Similar to aperture assembly 300 described above, the motor 920 may be coupled to a radial gear of the aperture assembly 900 via a drive belt, although alternate coupling mechanisms such as, for example, a gear driven system (e.g., one or more interconnecting gears) may be utilized. In addition, similar to aperture assembly 300 described above, the aperture assembly 900 may include first, second, third, and fourth motors for individually controlling each blade. In contrast to aperture assembly 300 described above, each motor may include a gear driven system 954, 956 for coupling to each blade. As such, lead screws may be replaced by a gear driven system including a first gear associated with each motor and a second gear or pinion associated with each blade.

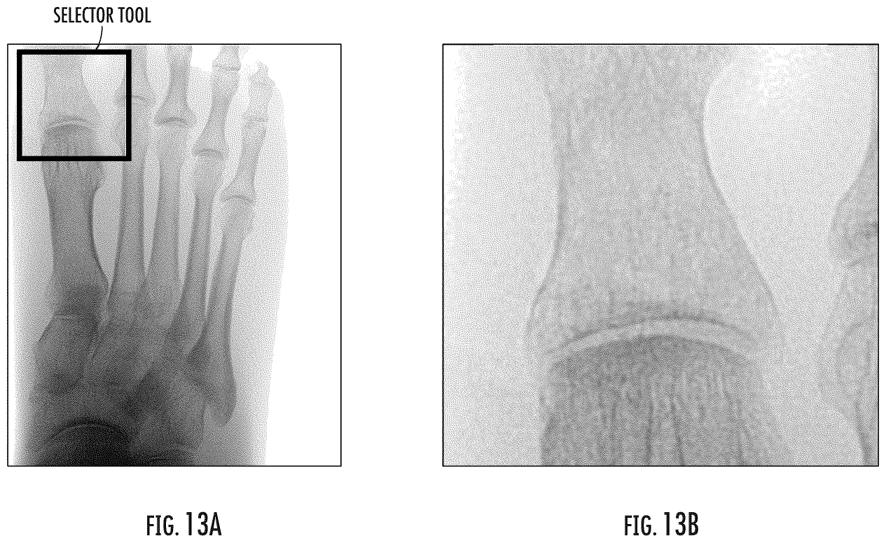

Referring to FIGS. 13A and 13B, in accordance with one or more features of the present disclosure, the aperture assembly may enable a custom zoom or magnification option. More particularly, the aperture assembly enables an operator to limit the X-ray beam to select and/or zoom in on a particular or desired area of the patient's anatomy (e.g., an operator can select any area for magnification within the initial X-ray view). For example, by utilizing and/or incorporating an aperture assembly in accordance with features of the present disclosure, the mini C-arm may enable the operator to select a region of interest and then limit the field of view to the selected region of interest.

A user interface such as, for example, a control panel may be mounted on the X-ray source or detector (i.e., at one end or the other of the C-arm assembly) and coupled to a computer of the system to provide a physician with easy access within the sterile field to imaging control functions associated with the system. The control panel may include an array of switches or buttons which enable the operator to select a region of interest by cycling through sections of an image or flipping through a series of images. In addition to or as an alternative to the control panel on the X-ray source or detector, the user interface may include a foot pedal with an array of foot switches to enable the operator to cycle through different areas of an image to select the region of interest. Other alternative user interfaces may include, for example, a keyboard or touch screen which may enable the operator to select a region of interest by, for example, drawing, sketching, outlining, etc., a particular area of an image.

An initial X-ray image may be taken of the patient's anatomy (FIG. 13A). The initial X-ray image may be taken without collimating the X-ray beam (i.e., with aperture assembly having a maximum aperture size), and the entire image may be displayed on a monitor or screen. If the operator (e.g., surgeon) desires to focus on a particular portion of the patient's anatomy, the operator may select a region of interest in the initial X-ray image and limit the field of view to the selected region of interest. The operator may select the region of interest using a user interface such as, for example, a control panel or foot pedal or the like. The blades of the aperture assembly may then be automatically adjusted utilizing the independently controlled aperture blades to limit the X-ray beam to an area on the detector corresponding to the selected region of interest, and an image may then be acquired. By enabling independent control over the blades in the aperture assembly, the field of view may be adjusted to focus on the portion of the patient's anatomy that is of interest to the clinician (FIG. 13B). In certain embodiments, the operator may desire a magnified image of the region of interest. In these embodiments, the acquired image may be digitally magnified and displayed without removing the patient's anatomy from the detector. The level of magnification may be inputted by the user or selected based one or more pre-set magnifications that are optimized for the portion of the anatomy being imaged. It is contemplated that the magnification step may occur automatically upon adjustment of the field of view in a one-step sequence or sequentially upon input from the operator.

In addition to providing complete freedom to select a particular area of interest, Mag view enables a high-quality X-ray image to be obtained while simultaneously reducing the X-ray dose experienced by the patient and/or operator. This is in contrast to current magnification operations which only enable two views: a large view and a small view (e.g., current mini C-arms do not provide freedom to select a particular area of interest but rather only allow an operator to select between a large view or area or a smaller, magnified view or area). Alternatively, current technologies require the operator to move the patient's anatomy closer to the source in order to obtain a magnification view, which not only increases the radiation hazard to the patient but also requires repositioning the patient's anatomy off of the detector toward the source.

In addition, in accordance with one or more features of the present disclosure, the aperture assemblies may also include one or more limit switches. For example, a first limit switch may be associated with the rotational mechanism (e.g., the motor and the drive belt and pulley system or the gear driven system). The limit switch may detect an end of travel for rotation of the aperture assembly. Thus arranged, during calibration, the limit switch provides a home location for calibration of the aperture assembly. In addition, and/or alternatively, each of the blades in the aperture assemblies may include or be associated with a limit switch. For example, in one embodiment, each blade may include a limit switch positioned outside of the blades. The limit switches may detect a home position for each of the blades. Thus arranged, during calibration, the limit switches can be utilized to detect a home position for each of the blades.

The source 252 and the detector 254 may be any suitable source and detector now known or hereafter developed. For example, the source 252 may be, for example, an X-ray source. The detector 254 may be, for example, a flat panel detector including, but not limited to, an amorphous silicon detector, an amorphous selenium detector, a plasma-based detector, etc. The source 252 and detector 254 create an image of a patent's anatomy, such as for example a hand, a wrist, an elbow, a foot, etc.

While the present disclosure makes reference to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the embodiments or configurations of the disclosure may be combined in alternate embodiments or configurations. Moreover, the following claims are hereby incorporated into this Detailed Description by this reference, with each claim standing on its own as a separate embodiment of the present disclosure.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A mini C-arm imaging apparatus comprising:
a C-arm assembly;
a movable base; and
an arm assembly coupling the C-arm assembly to the movable base;
wherein the C-arm assembly includes:
a first end, a second end, and a curved intermediate body portion extending between the first and second ends, the C-arm assembly including an X-ray source adjacent the first end and a detector at the second end, the curved intermediate body portion defines an arc length extending between the first and second ends, the X-ray source being movable along the arc length of the curved intermediate body portion and relative to the detector to enable the mini C-arm to acquire a first image when the X-ray source is at a first position on the curved intermediate body portion and a second image when the X-ray source is at a second position on the curved intermediate body portion, the second position being different that the first position, so that the first and second images of the patient's anatomy are taken at different angles relative to the patient's anatomy and are acquired without moving the patient's anatomy; and
an aperture assembly operatively coupled to the X-ray source such that an X-ray beam passes from the X-ray source through the aperture assembly and onto the detector, the aperture assembly comprising:
first, second, third, and fourth blades defining an aperture, the first and second blades positioned on opposing sides of the aperture, the third and fourth blades positioned on opposing sides of the aperture and orthogonal to the first and second blades, each of the first, second, third, and fourth blades being independently controlled to adjust a field of view.

2. The mini C-arm imaging apparatus according to claim 1, wherein the aperture assembly includes first, second, third, and fourth motors operatively coupled to the first, second, third, and fourth blades, respectively.

3. The mini C-arm imaging apparatus according to claim 2, wherein the aperture assembly further includes first, second, third, and fourth lead screws, the first lead screw coupling the first motor to the first blade, the second lead screw coupling the second motor to the second blade, the third lead screw coupling the third motor to the third blade, and the fourth lead screw coupling the fourth motor to the fourth blade.

4. The mini C-arm imaging apparatus according to claim 1, wherein the aperture assembly includes a rotational motor and a drive belt, the drive belt extending between the rotational motor and a radial gear of the aperture assembly so that activation of the rotational motor rotates the aperture assembly relative to the X-ray source.

5. The mini C-arm imaging apparatus according to claim 4, wherein the aperture assembly includes a position sensing system to detect movement of the X-ray source, upon detection of movement of the X-ray source, the rotational motor automatically rotates the aperture assembly to match movement of the X-ray source.

6. The mini C-arm imaging apparatus according to claim 4, wherein the aperture assembly includes a plurality of bearings to ride in a corresponding groove formed in the aperture assembly to guide rotation of the aperture assembly.

7. The mini C-arm imaging apparatus according to claim 1, wherein the aperture assembly includes a sensor to detect a position of each of the first, second, third, and fourth blades.

8. The mini C-arm imaging apparatus according to claim 7, wherein the aperture assembly includes a first subassembly and a second subassembly, the first subassembly including the first and second blades, the second subassembly including the third and fourth blades.

9. The mini C-arm imaging apparatus according to claim 8, wherein the sensor is an inductance sensor PCB positioned between the first subassembly and the second subassembly.

10. The mini C-arm imaging apparatus according to claim 9, wherein the sensor PCB includes an upper surface, a lower surface, and first, second, third, and fourth coils, the first and second coils positioned in the upper surface and in alignment with the first and second blades, respectively, the third and fourth coils positioned in the lower surface and in alignment with the third and fourth blades, respectively, and wherein each of the first, second, third, and fourth blades includes a target such that movement of the blades causes a respective target to move relative to a respective coil creating a resulting electromagnetic field.

11. The mini C-arm imaging apparatus according to claim 1, wherein the aperture assembly includes a pre-collimator and a filter to attenuate the incoming X-ray beam, the pre-collimator reducing a size of the incoming X-ray beam before passing through the aperture.

12. The mini C-arm imaging apparatus according to claim 1, wherein the aperture assembly enables a custom magnification view to enable an operator to adjust the size of the X-ray beam emitted from the X-ray source to select a desired field of view.

13. The mini C-arm imaging apparatus according to claim 1, wherein the first and second images of the patient's anatomy are different radiographic views of the patient's anatomy.

14. The mini C-arm imaging apparatus according to claim 1, wherein the first and second images of the patient's anatomy are combined into a three-dimensional rendering of the patient's anatomy.

15. A method for generating a custom magnification image of a patient's anatomy using a mini C-arm, the mini C-arm including an aperture assembly having independently controlled aperture blades, the method comprising:
taking an initial X-ray of the patient's anatomy positioned on a detector of the mini C-arm;
selecting a region of interest of the patient's anatomy;
adjusting the independently controlled aperture blades of the mini C-arm to focus an emitted X-ray beam to the selected region of interest; and
digitally magnifying the selected region of interest to generate a magnified image of the patient's anatomy without removing the patient's anatomy from the detector.

16. The method of claim 15, wherein digitally magnifying the selected region of interest is performed automatically.

17. The method of claim 15, wherein digitally magnifying the selected region of interest is performed upon input from an operator.

18. The method of claim 15, wherein digitally magnifying the selected region of interest comprises magnifying the image by a percentage input by the user.

19. The method of claim 15, wherein digitally magnifying the selected region of interest comprises a percent magnification, the percent magnification being a pre-set magnification based on the anatomy being imaged.

20. The method of claim 15, wherein selecting a region of interest of the patient's anatomy comprises using a user-interface including a control panel operatively coupled to a computer system, the control panel including an array of switches or buttons to enable an operator to select the region of interest by cycling through sections of the initial X-ray or flipping through a series of images taken from the initial X-ray.

21. The method of claim 15, wherein selecting a region of interest of the patient's anatomy comprises a foot pedal operatively coupled to a computer system, the foot switches including an array of foot switches to enable an operator to cycle through different areas of the initial X-ray to select the region of interest.

22. The method of claim 15, wherein selecting a region of interest of the patient's anatomy comprises one of a keyboard, a touch screen, or a combination thereof, to enable an operator to select the region of interest.

23. The method of claim 15, wherein taking an initial X-ray of the patient's anatomy positioned on a detector of the mini C-arm comprises taking an image of a full-view of the patient's anatomy.
